(19) United States
(12) Patent Application Publication
(10) Pub. No.: US 2007/0068526 A1
(43) Pub. Date: Mar. 29, 2007

(54) Device for supplying a respiratory gas and air-conduction structure provided in said device

(76) Inventors: Bernd Lang, Grafelfing (DE); Achim Biener, Aufkirchen (DE); Wolfgang Mayer, Ebringen (DE); Jahann Burz, Wielenbach/Wildzhofen (DE); Martin Bechtel, Winsen/Luhe (DE); Jorn Lange, Siegen (DE)

Correspondence Address: NIXON & VANDERHYE, PC, 901 NORTH GLEBE ROAD, 11TH FLOOR, ARLINGTON, VA, 22203, US

(21) Appl. No.: 10/578,491

(22) PCT Filed: Nov. 5, 2004
(86) PCT No.: PCT/EP04/12565

§ 371 Date: Jul. 6, 2006

(30) Foreign Application Priority Data
Nov. 5, 2003 (DE) 103 51 503.8

(51) Int. Cl. A61M 16/00; A62B 7/00; F16K 31/02

(52) U.S. Cl. 128/204.22; 128/204.18

(57) ABSTRACT

The invention relates to a device for supplying a respiratory gas, in particular a CPAP device and also to an air-conduction structure that is provided in said device. The aim of the invention is to provide a device of this type for supplying a respiratory gas, which is characterised by a silent operation and which has advantages in terms of the assembly method and of hygiene over conventional devices of this type. This is achieved by a device for supplying a respiratory gas, comprising a transport unit for transporting said gas at a pressure level that exceeds the ambient pressure, a housing for receiving the transport device and an air-conduction structure for guiding the respiratory gas from the transport device to an outlet region, said air-conduction device being configured by a moulded foam part comprising respiratory-gas channels. This enables the provision of a respiratory-gas supply device, in particular a CPAP device, in which the air-conduction device is formed by a sound absorbing, interchangeable foam element, or a foam element that can be washed separately.

The invention relates to a device for supplying a respiratory gas, and in particular relates to a CPAP device. The invention also relates to an air-conduction conduit per se, provided in such a CPAP device.

In CPAP devices, typically the respiratory gas supplied to a patient is delivered by a delivery device at a pressure level that is above the ambient pressure. This delivery device may in particular be embodied as a blower device, with a motor-driven impeller in the form of an axial, semiaxial, or radial impeller. Depending on the design of the CPAP device, it is possible for the delivery device, given a suitable counterpressure, to have a flow through it counter to its delivery direction in some phases as well. Hence fundamentally the delivery device forms a pressure gate, by means of which the air-conduction system toward the patient is at a higher pressure than the air-conduction system that is open to the environment. The degree of air backflow during the expiratory phase is determined essentially by the inhalation volume as well as derivation effects. The supply of the respiratory gas to a user can be done via a breathing mask device, which is joined to the CPAP device by way of a flexible hose. Labyrinth portions may be embodied in the interior of the CPAP device, for absorbing any acoustic events coupled into the respiratory gas by the delivery device. These labyrinth portions may be lined with a sound absorbing material, in order to enhance the sound absorption capacity of the labyrinthine path.

The object of the invention is to create a device of the type defined at the outset for supplying a respiratory gas which is distinguished by extremely quiet operation and offers advantages, in terms of assembly as well as hygiene, over conventional devices of this type.

This object is attained according to the invention by a device for supplying a respiratory gas, in particular a CPAP device, having a delivery device for delivering the respiratory gas at a pressure level that is above the ambient pressure, a housing device, for receiving the delivery device, and an air-conduction structure for conducting the respiratory gas from the delivery device to an outlet region, wherein the air-conduction structure is embodied as a molded foam part made from a foamed material.

A housing device for receiving the delivery device, and an air-conduction structure for conducting the respiratory gas from the delivery device to an outlet region, in which the air-conduction structure is formed by a molded foam part with respiratory gas conduits embodied in it.

As a result, it advantageously becomes possible to create a respiratory gas supplying device, in particular a CPAP device, in which an air-conduction structure is formed directly by a sound absorbing foam element that either is interchangeable or can be separately washed out or washed off.

Preferably, the molded foam part is subdivided into a first portion of the molded part and a second portion of the molded part. The two portions of the molded part are preferably designed here such that they can be put together via boundary faces that are complementary to one another. The two portions of the molded part may have intermeshing portions, which enable positioning the portions relative to one another or also define certain conduit wall portions. The two portions of the molded part may also be attached to a common intermediate structure.

The air-conduction conduit is preferably embodied such that it forms a sound absorption path. The sound absorption path may be formed upon the cooperation of the first portion of the molded part with the second portion of the molded part; various parts of the conduit wall are formed in alternation, or supplementing one another, by corresponding wall portions of the two portions of the molded part. It is also possible to embody the air-conduction structure such that a first portion of the airway is formed by the first portion of the molded part and then successively in portions by the second portion of the molded part.

Preferably, support structures are provided, for bracing the molded foam part. These support structures may, in the form of platelike, tubular, pinlike or needlelike elements, penetrate the molded foam part or recesses provided therein and fix and reinforce the molded foam part.

It is possible to couple the molded foam part detachably to the support structures. This makes it possible to replace the molded foam part in a simple way during a maintenance operation. It is also possible to embody both the CPAP device and the molded foam part such that the molded foam part can advantageously be replaced as a disposable item.

The molded foam part can be embodied such that it is solidly joined to the support structures, for instance embodied integrally with them, or is integrally molded onto them or glued to them. The molded foam part thus provided with support structures can also be embodied as a replaceable and in particular a disposable component. The support structures may take on additional functions, such as fixing or lining the molded foam part, where they may form a component of the respiratory gas line path or of connection structures.

In a special aspect of the present invention, the molded foam part is embodied such that it defines a receiving portion, for elastically resiliently receiving the delivery device. As a result, the delivery device can advantageously be coupled with the molded foam part in an acoustically encapsulated way and in particular can be stored that way.

The receiving portion may be embodied such that the delivery device is received in it without play, with a slight press fit. It is possible for the delivery device to be elastically suspended, braced or stored via regions of a housing device that forms a component of it, or in cooperation with the motor. The contact faces of the molded foam part that enter into contact with the delivery device may be adapted to the external geometry of the portions of the delivery device embedded in it. It is possible to accomplish a largely full-surface-area reception of the delivery device. It is also possible, in the region of the contact zone between the molded foam part and the delivery device, to provide recesses, indentations, or conduit furrows, so that in some portions, there is no direct contact between the molded foam part and the delivery device. Via the interstices thus formed, a defined flow of cooling air can be made possible. The cooling air flowing through these regions may be diverted from an overpressure region of the respiratory gas path. The conduction of the cooling air is preferably accomplished such that no reaspiration or return of the cooling air, or of air otherwise coming into contact with electrical or electronic components, into the respiratory gas path course can take place.

Storing the delivery device in the molded foam part can also be done by including the support structures in the process. These support structures may be embedded in the molded foam part.

It is possible to embody the molded foam part such that the first portion of the molded part and the second portion of the molded part have different material properties. As a result, by adaptation of the material properties, it becomes possible to achieve especially favorable sound absorption effects. It is also possible, via the molded foam part, to realize a humidifying device, by making at least one of the portions of the molded part of a material suitable as a humidifying diaphragm.

It is also possible, in cooperation with the molded foam part, to realize a filter device, by using at least one of the portions of the molded part to form a filter wall. It is also possible to attach a filter layer (for instance of fabric, filter paper, or cellulose, or the like) or a further foam body to the molded foam part in sandwichlike fashion.

The foam body, in an especially preferred embodiment of the invention, forms a portion to stand on, by way of which the CPAP device can be braced on a surface to stand on in a way that is advantageous in terms of minimized transmission of structure-borne sound. The portion to stand on may be formed by foot zones that protrude slightly downward. The dimensions of the foot zones are preferably adapted such that under the intrinsic weight of the CPAP device, a defined compression and hence prestressing of the foam material results. The degree of the prestressing can be adapted such that residual vibration typical of the CPAP device can be cancelled or isolated with high probability or effectiveness.

In an especially preferred embodiment of the invention, the CPAP device has a housing device, embodied in the form of a receiving bell, which is placed fittingly on the foam body and surrounds the foam body.

It is possible to design the molded foam part, or the foam body formed by it, such that at least some of the air-conduction conduits are formed by an outer surface region of the foam body. The sound absorption path preferably has a multiply winding course. The inner wall of the conduit, which surrounds the sound absorption path and is formed by the foam body or a coating provided on it, can be provided with sound absorbing profile sections. It is possible to equip the foam material, or at least the wall regions that come into contact with the respiratory gas, with germ-suppressing additives, hygienic materials, disposable inlays, and antimicrobial materials, in particular nanosilver dopants.

The object stated at the outset is also attained by a CPAP device, including a core module and an outer module provided for receiving the core module, in which the core module has a foam body, and an air-conduction path is embodied in the foam body and is in communication with a respiratory gas delivery device.

The respiratory gas delivery device is preferably embedded in the foam body. The foam body is preferably embodied in multiple parts. Function components may advantageously be inserted into the foam body. In particular, it is possible to embed or insert conduction structure components into the foam body. The conduction structure component may in particular be embodied as a breathing hose connection structure component.

It is possible, by means of the foam body, to furnish a securing device for at least partly suspending the delivery device and/or other function components, in particular a power pack of the CPAP device.

It is possible to insert further function components, carried by the molded foam part, in the form of sensor devices for detecting a pressure of the respiratory gas and/or the respiratory gas volumetric flow.

The other devices and function components inserted at least partly into the molded foam part may also be valve devices, switch devices, and other kinds of sensor devices.

The air-conduction paths may be realized on the basis of insert or shell concepts. Interfaces may be formed by the molded foam part or by structures inserted into it.

It is possible for certain function components, such as parts of a blower housing, a bottom unit, sensor device portions, sensor carrier devices, and line connection portions to be embodied such that, after the molded foam part is put on or attached, they communicate with the corresponding portions of the respiratory gas path system or are positionally correctly braced, secured or suspended in some other way by the molded foam part.

The molded foam part may be made from a styrofoam-like foam material, an elastomer foam material, in particular LSR (liquid silicone rubber), neoprene material, foamed thermoplastics, and foamed rubber or natural rubber materials, in particular silicone rubber materials. The molded foam part may be embodied with open or closed cells. The pore size can be largely homogeneous, or—to achieve defined load-bearing properties—nonhomogeneous. The molded foam part can also be embodied with a skin either locally—for instance in the region of the respiratory gas line portions—or over the full surface. The application of skin can be done by applying films, immersion, spraying, or in-mold coating. The molded foam part may embodied as a largely rigid part or as a hard-foamlike soft part.

Microprofiles, in particular microdomes, may be embodied in the region of the respiratory gas paths, for the sake of sound absorption.

Further details and characteristics of the invention will become apparent from the ensuing description in conjunction with the drawings. Shown are:

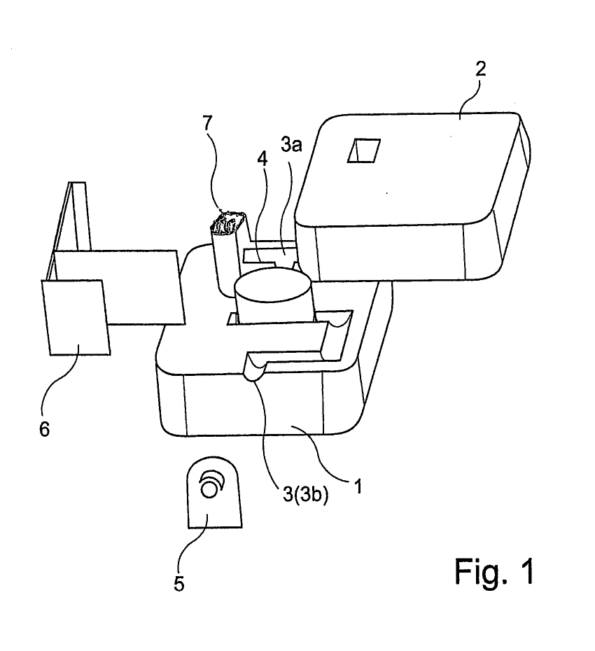

FIG. 1, a perspective exploded view of a two-part molded foam part, as well as the function components cooperating with it, in the form of a blower device, a connection structure component, a suction filter, and support wall inserts;

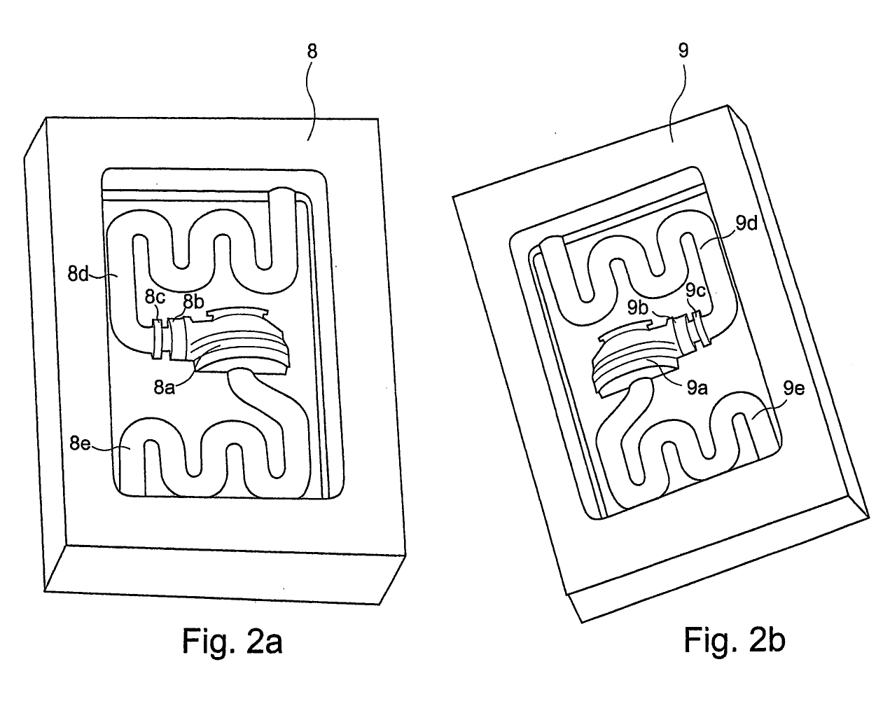

FIG. 2a, a perspective view of a first molding tool shell for producing a first portion of the molded part of the molded foam part of the invention;

FIG. 2b, a perspective view of a second molding tool shell for producing a second portion of the molded part of the molded foam part;

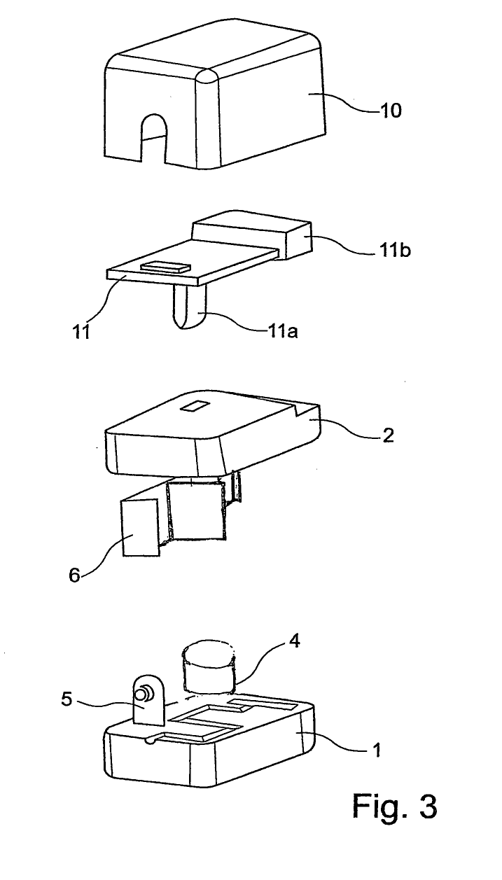

FIG. 3, a perspective exploded view of the components of FIG. 1 in combination with an external housing bell;

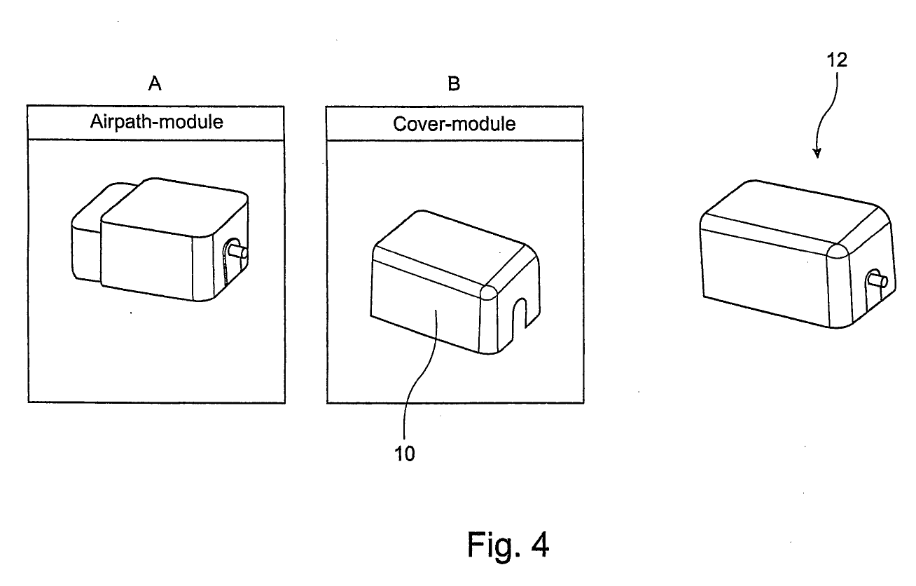

FIG. 4, a view for explaining a modular concept that can be realized in accordance with the invention;

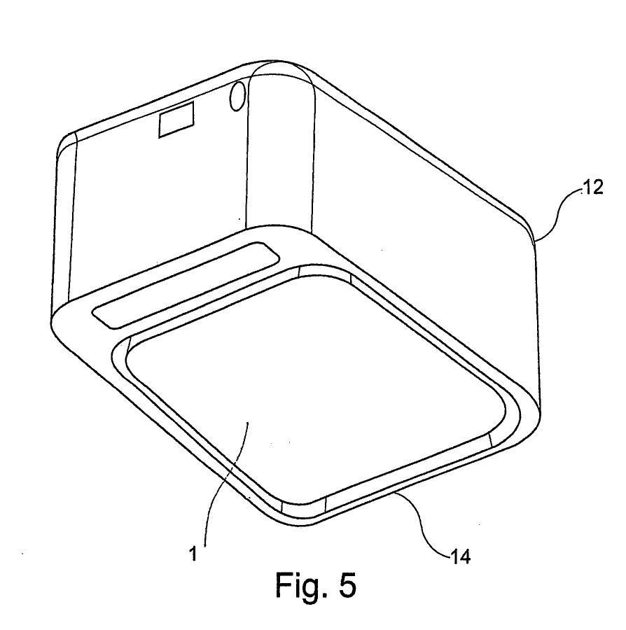

FIG. 5, a perspective view of the CPAP device of FIG. 4 from below, for explaining the bottom part formed by the molded foam part;

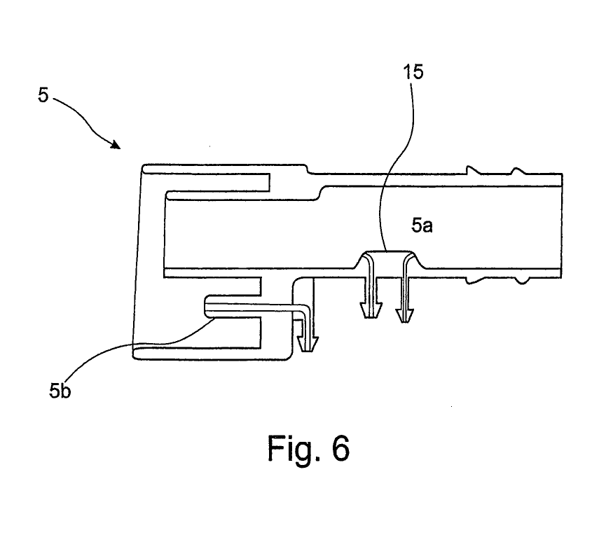

FIG. 6, a sketch for explaining a connection structure component, embedded in some portions in the molded foam part, with a pressure measuring port and a breathing flow sensor portion;

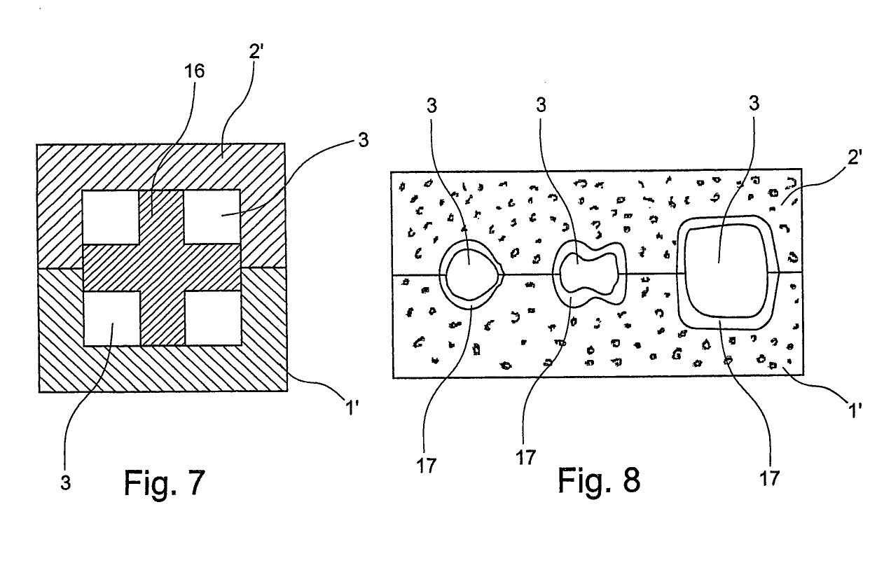

FIG. 7, a sketch for explaining a construction concept for furnishing the respiratory gas line portions by means of a multi-part molded foam part;

FIG. 8, a sketch for explaining various conduit cross sections formed by foam conduit inlays.

FIG. 1 shows a perspective exploded view of a molded foam part, subdivided into a first portion of the molded part 1 and a second portion of the molded part 2, with air-carrying conduits 3.

A blower device 4 is inserted into the first portion of the molded part 1, and through it respiratory gas is aspirated via an inlet line portion 3a and delivered at a predetermined overpressure level into an outlet portion 3b of the air-conduction conduit.

The inlet line portion 3a and the outlet portion 3b have a multiply winding course, so that as a result, an especially high sound absorption capacity is attained. The two-part molded foam part can be coupled to a connection structure component 5, and the connection structure component 5 is embodied such that it forms an interface device, for the connection of a respiratory gas hose.

A support structure 6 can also be inserted into the molded foam part; on the one hand, it reinforces the molded foam part, and on the other, it partitions certain zones of the molded foam part off from one another with increased tightness. A plug-in module 7 can also be inserted into the molded foam part, and by way of it special additional functions can be realized, such as monitoring, control, and filter functions.

The first portion of the molded part 1 and the second portion of the molded part 2 are embodied such that they can be put together in complementary fashion, leaving required function-relevant interstices, in particular the respiratory gas conduits.

FIG. 2a shows a perspective view of a first molding tool shell 8 for producing a first portion of the molded part of the molded foam part of the invention.

FIG. 2b shows a perspective view of a second molding tool shell 9 for producing the second portion of the molded part of the molded foam part. Molding wall structures are embodied in the two molding tool shells 8, 9, and by way of them, the recesses required for both receiving the respiratory gas delivery device and for forming the respiratory gas line paths are formed in the respective portion of the molded part in the foam material to be placed in the mold. The mold wall portions 8a, 9a shown in FIGS. 2a, 2b, respectively, each serve to form the recess intended for receiving the blower device 4 (see FIG. 1). The wall sections 8b, 8c on the one hand and 9b, 9c serve to form recesses in the respective portion of the molded part for receiving coupling cuffs.

The wall portions 8d, 9d serve to form the outlet line portions of the air-carrying conduits that extend inside the molded foam part. The wall portions 8e, 9e serve to form the inlet line portions of the air-carrying conduits. The air-carrying conduits have a multiply winding course on both the suction side and the compression side.

FIG. 3, in the form of an exploded view, shows the components already described in conjunction with FIG. 1, along with a housing bell 10 intended for receiving them. The housing bell 10 is dimensioned such that it can be placed with a slight press fit on the molded foam part, formed of the first portion of the molded part 1 and the second portion of the molded part 2. A further insert element 11 is provided in an interstice defined between the top of the housing bell 10 and the molded foam part; this insert element may for instance be electrical components of the CPAP device, in particular a control circuit board, and a power pack arrangement. Particularly if the further insert element is designed as a control component, it is possible to equip it directly with sensor devices 11a for detecting control-relevant parameters. A filter body 11b is also received in the aforementioned interstice.

FIG. 4, in a schematic drawing, shows a CPAP device 12, constructed in modular fashion on the basis of the molded foam part of the invention. This CPAP device 12 includes the component unit A, called an airpath module and essentially comprising the molded foam part and the components received in it, and the component unit B, here called a cover module, which includes both the housing bell 10 (FIG. 3) and the electrical circuit devices (not shown) received in it. The modules A and B may be embodied such that by suitably placing the cover module on the airpath module, a suitably configured CPAP device is available.

By means of the molded foam part of the invention, bottom structures of the CPAP device can also be realized, as is illustrated in FIG. 5.

By means of the first portion of the molded part 1 of the molded foam part, it is possible to realize a structure 14 to stand on, by way of which the CPAP device 12 can be stood elastically resiliently on a surface to stand on. The structure 14 to stand on is embodied as a peripheral bead, in the exemplary embodiment shown here.

FIG. 6 shows a preferred embodiment of a connection structure component 5 which can be inserted into the molded foam part of the invention and which has a respiratory gas line portion 5a and a pressure measuring hose connection portion 5b. The connection structure component 5 can be embedded in some portions in the molded foam part of the invention. On the connection structure component 5, a flow sensor device 15 is also provided, by which pressure signals from the line portion 5a can be picked up, on the basis of which signals the respiratory gas flow can be calculated.

FIG. 7 is a basic sketch for explaining other possibilities for embodying both the air-carrying conduits and the voids to be embodied in the molded foam part for receiving function components of the CPAP device. In the variant shown in FIG. 7, a core body 16, formed of a foam material, is inserted into both a first portion of the molded part 1′ and a second portion of the molded part 2′, leaving air-conduction conduits 3.

FIG. 8 shows a sketch of a molded foam part of the invention, with air-carrying conduits 3 of different cross-sectional geometries embodied in it. In this exemplary embodiment, the air-carrying conduits 3 are provided with a sheath 17, which here is likewise made from a foam material and which is inserted into the first and second portions of the molded part 1′, 2′.

1-32. (canceled)

33. A device for supplying a respiratory gas, in particular a CPAP device, having:
a delivery device for delivering the respiratory gas at a pressure level that is above the ambient pressure,
a housing device, for receiving the delivery device, and
an air-conduction structure for conducting the respiratory gas from the delivery device to an outlet region,
wherein the air-conduction structure is embodied as a molded foam part made from a foamed material.

34. The device in accordance with claim 33, characterized in that the molded foam part defines air-carrying conduits.

35. The device in accordance with claim 33, characterized in that the molded foam part is subdivided into a first portion of the molded part and a second portion of the molded part.

36. The device in accordance with claim 33, characterized in that the air-conduction structure is embodied such that it forms a sound absorption path.

37. The device in accordance with claim 33, characterized in that the sound absorption path is formed upon the cooperation of the first portion of the molded part with the second portion of the molded part.

38. The device in accordance with claim 33, characterized in that the sound absorption path is formed in some portions by the first portion of the molded part and in some portions by the second portion of the molded part.

39. The device in accordance with claim 33, characterized in that support structures are provided, for bracing the molded foam part.

40. The device in accordance with claim 33, characterized in that the molded foam part is detachably coupled to the support structures.

41. The device in accordance with claim 33, characterized in that the molded foam part is injection-molded onto the support structures.

42. The device in accordance with claim 33, characterized in that the molded foam part defines a receiving portion, for elastically resiliently receiving the delivery device.

43. The device in accordance with claim 33, characterized in that the receiving portion is embodied such that the delivery device is received in it without play, with a slight press fit.

44. The device in accordance with claim 33, characterized in that the first portion of the molded part and the second portion of the molded part have different material properties.

45. The device in accordance with claim 33, characterized in that at least one of the portions of the molded part forms a filter device.

46. The device in accordance with claim 33, characterized in that a filter device is coupled to the foam body.

47. The device in accordance with claim 33, characterized in that the foam body forms a portion to stand on.

48. The device in accordance with claim 33, characterized in that the housing device forms a receiving jacket and is placed onto the foam body.

49. The device in accordance with claim 33, characterized in that at least some of the air-conduction conduits are formed by an outer surface region of the foam body.

50. The device in accordance with claim 33, characterized in that the sound absorption path has a multiply winding course.

51. The device in accordance with claim 33, characterized in that the inner wall of the conduit, which surrounds the sound absorption path and is formed by the foam body or a coating provided on it, is provided with sound absorbing profile sections.

52. A CPAP device, including a core module and an outer module provided for receiving the core module, wherein the core module includes a foam body, and an air-conduction path is embodied in the foam body and is in communication with a respiratory gas delivery device, for furnishing a respiratory gas conduction portion with sound absorbing properties.

53. The CPAP device in accordance with claim 52, characterized in that the respiratory gas delivery device is embedded in the foam body.

54. The CPAP device in accordance with claim 52, characterized in that the foam body is embodied in multiple parts.

55. The CPAP device in accordance with claim 52, characterized in that function components are inserted into the foam body.

56. The CPAP device in accordance with claim 52, characterized in that conduction structure components are inserted into the foam body.

57. The CPAP device in accordance with claim 56, characterized in that the conduction structure component is embodied as a breathing hose connection structure component and/or as an air humidifier connection structure component.

58. The CPAP device in accordance with claim 52, characterized in that the foam body forms a securing device for suspending the delivery device and/or other function components of the CPAP device.

59. The CPAP device in accordance with claim 52, characterized in that the further function components are a power pack.

60. The CPAP device in accordance with claim 52, characterized in that the further function components are sensor devices for pressure and/or volumetric flow.

61. The CPAP device in accordance with claim 52, characterized in that the further function components are a control unit.

62. The CPAP device in accordance with claim 52, characterized in that the further function components are valve devices.

63. The CPAP device in accordance with claim 52, characterized in that the further function components are switch devices.

64. The CPAP device in accordance with claim 52, characterized in that the geometry of the foam part is determined by a plastic injection molding tool, and the foam part is produced by means of a plastic material injection molding operation.
